(19) United States
(12) Patent Application Publication
(10) Pub. No.: US 2016/0186230 A1
(43) Pub. Date: Jun. 30, 2016

(54) Enzyme Electrode

(71) Applicant: ARKRAY, Inc., Kyoto (JP)

(72) Inventor: Hisashi Kaneda, Kyoto (JP)

(73) Assignee: ARKRAY, Inc., Kyoto (JP)

(21) Appl. No.: 14/976,077

(22) Filed: Dec. 21, 2015

(30) Foreign Application Priority Data
Dec. 24, 2014 (JP) 2014-261203

(51) Int. Cl. C12Q 1/00; G01N 27/327

(52) U.S. Cl. CPC C12Q 1/006; G01N 27/3271; G01N 27/3272; G01N 27/3273

(57) ABSTRACT

The present invention provides an enzyme electrode comprising: an electrode; and a detection layer which is in contact with the electrode, where the detection layer includes an enzyme, a crosslinking agent, an electrically conductive polymer and a sugar, wherein electrons are transferred between the enzyme in the detection layer and the electrode.

### TECHNICAL FIELD

The present invention relates to an enzyme electrode for measuring a charge transfer limiting current.

### BACKGROUND ART

An enzyme electrode is known which comprises an electrode as a base material, and a detection layer in which molecules of an enzyme and electrically conductive particles are immobilized on the surface of the electrode, using a crosslinking agent or a binder. The enzyme electrode has a structure in which electrons generated by an enzyme reaction are transferred. Specifically, JP 2014-006154 A discloses an enzyme electrode in which the detection layer thereof contains an enzyme, electrically conductive particles and a crosslinking agent. Further, JP 2014-006155 A discloses an enzyme electrode in which the detection layer thereof contains an enzyme, electrically conductive particles, and an electrically conductive polymer.

### SUMMARY OF THE INVENTION

An enzyme electrode is sometimes used for measuring the concentration of a target substance contained in a trace amount in a sample. In order to measure the concentration of the trace amount of the target substance, the sensitivity of the measurement needs to be improved. However, the enzyme electrodes disclosed in JP 2014-006154 A and JP 2014-006155 A still have room for improvement in the measurement sensitivity.

Namely, one aspect of the present invention is to provide an enzyme electrode for measuring the concentration of a target substance quantitatively and with a high sensitivity.

In order to achieve the above mentioned objective, one aspect of the present invention adopts the following constitution.

Specifically, one aspect of the present invention relates to:
an enzyme electrode comprising:
an electrode; and
a detection layer which is in contact with the electrode and comprises an enzyme, a crosslinking agent, an electrically conductive polymer and a sugar;
wherein electrons are transferred between the enzyme in the detection layer and the electrode.

The above mentioned sugar is preferably at least one type of sugar selected from sugars containing 2 or more monosaccharide units. Further, the enzyme is preferably an oxidoreductase.

Another aspect of the present invention relates to a biosensor comprising the above mentioned enzyme electrode.

Further, another aspect of the present invention relates to:
a measuring apparatus comprising:
the above mentioned biosensor;
a control section configured to control the application of voltage to the biosensor;
a detection section configured to detect a charge transfer limiting current based on the transfer of electrons derived from a substance to be measured to the electrode, wherein the charge transfer limiting current is generated by the application of voltage to the biosensor;
an arithmetic section configured to calculate the concentration of the substance based on the value of the charge transfer limiting current; and
an output section configured to output the calculated concentration of the substance.

In addition, another aspect of the present invention relates to a method for producing an enzyme electrode comprising forming a detection layer by applying a mixed solution on an electrode, the mixed solution comprising an enzyme, a crosslinking agent, an electrically conductive polymer and a sugar; wherein electrons are transferable between the enzyme and the electrode in the enzyme electrode.

According to the present invention, an enzyme electrode having an improved measurement sensitivity and qualitative properties can be provided by incorporating a sugar in a detection layer of the enzyme electrode.

### BRIEF DESCRIPTION OF THE DRAWINGS

FIG. 1 is a schematic diagram illustrating the structure of an enzyme electrode according to one embodiment of the present invention.

FIG. 2 is a schematic diagram illustrating one embodiment of a measuring apparatus according to the present invention.

FIG. 3 is a flow chart illustrating one embodiment of a measurement program using the measuring apparatus according to the present invention.

FIG. 4 is a graph of the current ratio (S/B), with (S) or without (B) glucose, showing the case where the enzyme electrode comprising a detection layer which contains no sugar is used (Comparative Example 1); and the case where the enzyme electrode comprising a detection layer which contains sucrose is used (Example 1).

FIG. 5 is a graph of the current ratio (S/B), with (S) or without (B) glucose, showing the case where enzyme electrodes each comprising a detection layer containing one of various types of sugar are used.

### EMBODIMENTS FOR CARRYING OUT THE INVENTION

The enzyme electrode as one embodiment of the present invention will now be described with reference to the drawings. The embodiment to be described below is provided only for illustration purposes, and the present invention is not limited to the constitution of the following embodiment.

### (Structure of Enzyme Electrode)

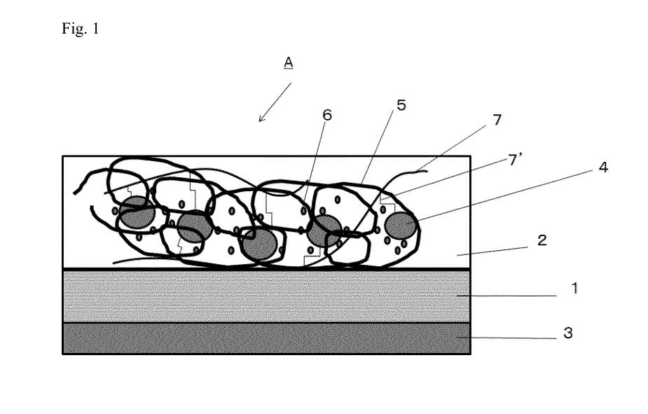

FIG. 1 is a schematic diagram of the enzyme electrode according to one embodiment of the present invention. In FIG. 1, an enzyme electrode A comprises an electrode 1; and a detection layer 2 formed on the surface of the electrode 1 (the upper surface, in FIG. 1).

### (Electrode)

The electrode 1 is made of a metallic material such as gold (Au), platinum (Pt), silver (Ag) or palladium (Pd); or a carbon material such as carbon. The electrode 1 is formed, for example, on an insulating base plate 3 as shown in FIG. 1. The insulating base plate 3 is made of an insulating material, and examples thereof include various types of resins (plastics), such as thermoplastic resins, for example, polyetherimide (PEI), polyethylene terephthalate (PET) and polyethylene (PE), polyimide resins, and epoxy resins; glasses; ceramics; papers; and the like. Any known material can be used as an electrode material for forming the electrode 1 and a material for forming the insulating base plate 3. The size and the thickness of the electrode 1 and the insulating base plate 3 can be determined as appropriate. Hereinafter, the combination of the insulating base plate 3 and the electrode 1 may be referred to as a “base material”.

### (Detection Layer)

The detection layer 2 is in contact with the electrode 1, and comprises an enzyme 4, an electrically conductive polymer 5, a sugar 6, and a crosslinking agent 7, but does not contain an electron mediator.

The enzyme electrode according to the present invention is used to measure a charge transfer limiting current based on the transfer of electrons derived from the substance to be measured to the electrode. The charge transfer limiting current is a current which is generated when the electrons are transferred from the enzyme to the electrode due to the reaction between the enzyme and the substance to be measured. Further, the charge transfer limiting current is a steady-state current which does not depend on time, and preferably, a steady-state current observed after the generation of a transient current due to the charging of an electric double layer.

In order to measure the charge transfer limiting current, it is preferred that a “direct electron transfer-type enzyme electrode” be used as a working electrode. The “direct electron transfer-type enzyme electrode” as used herein refers to a type of an enzyme electrode in which electrons are transferred between the enzyme and the electrode in such a way that electrons generated by an enzyme reaction in a reagent layer are directly transferred to the electrode (including the case in which the transfer of electrons is mediated by an electrically conductive polymer) without the involvement of an oxidation-reduction substance, such as an electron transfer mediator. In cases where an electron transfer mediator is used, if the molecules of the electron transfer mediator are immobilized so as not to be diffused, it is possible to measure the charge transfer limiting current.

As shown in FIG. 1, in the detection layer 2, the molecules of the enzyme 4 are crosslinked by the crosslinking agent 7, and further intertwined by the electrically conductive polymer 5 to exhibit a complex structure. The electrons generated by the enzyme reaction can be transferred to the electrode 1, directly or mediated by the electrically conductive polymer 5 which has an electrical conductivity. In other words, in the enzyme electrode A according to the embodiment of the invention, electrons are transferred between the enzyme 4 and the electrode 1 due to the direct electron transfer in the detection layer 2.

The limit distance within which the direct electron transfer could occur in a physiological reaction system is reported to be from 10 to 20 Å. In the electron transfer in an electrochemical reaction system comprising an electrode and an enzyme, the detection of the electron transfer on the electrode will be difficult if the distance between the electrode and the enzyme is longer than the above mentioned limit distance, unless it involves the transfer (for example, transfer by diffusion) of a mediator. Therefore, in the detection layer 2, the active sites of the enzyme 4 (the sites at which electrons are generated due to the enzyme reaction) and the electrically conductive sites of the electrically conductive polymer 5 are located within a distance suitable for electron transfer, in other words, the electrically conductive sites and the active sites are located close enough so that electrons are transferred therebetween in a suitable manner.

### (Enzyme)

Examples of the enzyme 4 include oxidoreductases. Examples of the oxidoreductase include: glucose oxidase (GOD), galactose oxidase, bilirubin oxidase, pyruvic acid oxidase, D- or L-amino acid oxidase, amine oxidase, cholesterol oxidase, choline oxidase, xanthine oxidase, sarcosine oxidase, L-lactic acid oxidase, ascorbic acid oxidase, cytochrome oxidase, alcohol dehydrogenase, glutamate dehydrogenase, cholesterol dehydrogenase, aldehyde dehydrogenase, glucose dehydrogenase (GDH), fructose dehydrogenase, sorbitol dehydrogenase, lactate dehydrogenase, malate dehydrogenase, glycerol dehydrogenase, 17β hydroxysteroid dehydrogenase, estradiol 17β dehydrogenase, amino acid dehydrogenase, glyceraldehyde 3-phosphoric acid dehydrogenase, 3-hydroxysteroid dehydrogenase, diaphorase, cytochrome oxidoreductase, catalase, peroxidase, glutathione reductase, and the like. Among others, the enzyme 4 is preferably a saccharide oxidoreductase. Examples of the saccharide oxidoreductase include: glucose oxidase (GOD), galactose oxidase, glucose dehydrogenase (GDH), fructose dehydrogenase, and sorbitol dehydrogenase.

Further, the oxidoreductase can contain at least one of pyrroloquinoline quinone (PQQ) and flavin adenine dinucleotide (FAD), as a catalytic subunit and a catalytic domain. Examples of the oxidoreductase containing PQQ include PQQ glucose dehydrogenase (PQQGDH). Examples of the oxidoreductase containing FAD include cytochrome glucose dehydrogenase (Cy-GDH) and glucose oxidase (GOD), having an α-subunit containing FAD. In addition, the oxidoreductase can contain an electron transfer subunit or an electron transfer domain. Examples of the electron transfer subunit include a subunit containing heme which has a function of giving and receiving electrons. Examples of the oxidoreductase having the subunit containing heme include those containing cytochrome. For example, a fusion protein of glucose dehydrogenase or PQQGDH with cytochrome can be used. Further, examples of the enzyme containing the electron transfer domain include cholesterol oxidase and quinoheme ethanol dehydrogenase (QHEDH (PQQ Ethanol dh)). As the electron transfer domain, it is preferred to use a domain containing cytochrome containing heme which has a function of giving and receiving electrons. Examples thereof include “QHGDH” (fusion enzyme; GDH with heme domain of QHGDH)), sorbitol dehydrogenase (Sorbitol DH), D-fructose dehydrogenase (Fructose DH), Glucose-3-Dehydrogenase derived from Agrobacterium tumefasience (G3DH from Agrobacterium tumefasience), and cellobiose dehydrogenase. A fusion protein of PQQGDH with cytochrome, which is an example of the above mentioned subunit containing cytochrome, and a cytochrome domain of PQQGDH, which is an example of the domain containing cytochrome, are disclosed, for example, in WO 2005/030807. Further, as the oxidoreductase, it is preferred to use an oligomeric enzyme comprising at least a catalytic subunit and a subunit containing cytochrome containing heme which has a function as an electron acceptor.

### (Electrically Conductive Polymer)

Examples of the electrically conductive polymer 5 include: polypyrrole, polyaniline, polystyrene sulfonate, polythiophene, polyisothianaphthene, polyethylenedioxythiophene(poly(3,4-ethylenedioxythiophene)poly(styrene sulfonate)), and combinations thereof. Examples of the commercially available product thereof, specifically, examples of the commercially available product of polypyrrole include: “SSPY” (ethyl 3-methyl-4-pyrrolecarboxylate) (manufactured by KAKEN INDUSTRY Co., Ltd.). Examples of the commercially available product of polyaniline include “AquaPASS 01-x” (manufactured by TA Chemical Co., Ltd.), and the like. Examples of the commercially available product of polystyrene sulfonate include “Poly-NaSS” (manufactured by TOSOH ORGANIC CHEMICAL CO., LTD.). Examples of the commercially available product of polythiophene include “Espacer 100” (manufactured by TA Chemical Co., Ltd.). Examples of the commercially available product of polyisothianaphthene include “Espacer 300” (manufactured by TA Chemical Co., Ltd.)). Examples of the commercially available product of polyethylenedioxythiophene(poly(3,4-ethylenedioxythiophene)poly(styrene sulfonate)) include “PEDOT-PSS” (manufactured by Polysciences Inc.).

Further, as the electrically conductive polymer 5, an electrically conductive polymer having various attributes (for example, water solubility) can be used. It is preferred that the electrically conductive polymer 5 contain a hydroxyl group or a sulfo group as a functional group.

### (Sugar)

The sugar 6 is a sugar which does not serve as a substrate for the enzyme 4. The sugar 6 contains, for example, 1 to 6 constituent sugars, preferably, 2 to 6 monosaccharide units. The sugar 6 may be a D-sugar or L-sugar, or a mixture thereof, and these can be used alone or in a combination of two or more. However, in cases where a sugar such as glucose is the target to be measured, a sugar which is different from the sugar to be measured, and which does not serve as a substrate for the enzyme 4 is used as the sugar 6.

Examples of disaccharides include: xylobiose, agarobiose, carabiose, maltose, isomaltose, sophorose, cellobiose, trehalose, neotrehalose, isotrehalose, inulobiose, vicianose, isoprimeverose, sambubiose, primeverose, solabiose, melibiose, lactose, lycobiose, epicellobiose, sucrose, turanose, maltulose, lactulose, epigentibiose, robinobiose, silanobiose, rutinose, and the like. Examples of trisaccharides include: glucosyl trehalose, cellotriose, chacotriose, gentianose, isomaltotriose, isopanose, maltotriose, manninotriose, melezitose, panose, planteose, raffinose, solatriose, umbelliferose, and the like. Examples of tetrasaccharides include maltosyl trehalose, maltotetraose, stachyose, and the like. Examples of pentasaccharides include maltotriosyl trehalose, maltopentaose, verbascose, and the like. Examples of hexasaccharides include maltohexaose and the like.

### (Crosslinking Agent)

Examples of the crosslinking agent 7, specifically, examples of aldehyde group-containing compounds to be used as the crosslinking agent 7 include: glutaraldehyde, formaldehyde, malonaldehyde, terephthalaldehyde, isobutyraldehyde, valeraldehyde, isovaleraldehyde, cinnamaldehyde, nicotinaldehyde, glyceraldehyde, glycoaldehyde, succinaldehyde, adipaldehyde, isophthalaldehyde, terephthalaldehyde, and the like. Examples of carbodiimide group-containing compounds include hexamethylene diisocyanate, hydrogenated xylylene diisocyanate, xylylene diisocyanate, 2,2,4-trimethyl hexamethylene diisocyanate, 1,12-diisocyanate dodecane, norbornane diisocyanate, 2,4-bis-(8-isocyanate octyl)-1,3-dioctyl cyclobutane, 4,4′-dicyclohexylmethane diisocyanate, tetramethyl xylylene diisocyanate, isophorone diisocyanate, and the like. The carbodiimide group-containing compounds are commercially available under the names of: CARBODILITE™ V-02, CARBODILITE™ V-02-L2, CARBODILITE™ V-04, CARBODILITE™ V-06, CARBODILITE™ E-02, CARBODILITE™ V-01, CARBODILITE™ V-03, CARBODILITE™ V-05, CARBODILITE™ V-07 and CARBODILITE™ V-09 (manufactured by Nisshinbo Chemical, Inc.). Examples of maleimide group-containing compounds include: m-maleimidobenzoyl-N-hydroxysuccinimide ester, sulfosuccinimidyl 4-(p-maleimidophenyl)butyrate, m-maleimidobenzoyl sulfosuccinimide ester, N-γ-maleimidobutyryloxy succinimide ester, succinimidyl 4-(N-maleimidomethyl)cyclohexane)1-carboxylate, N-succinimidyl-2-maleimidoacetic acid, N-succinimidyl-4-maleimidobutyric acid, N-succinimidyl-6-maleimidohexanoic acid, N-succinimidyl-4-maleimidomethyl cyclohexane-1-carboxylic acid, N-succinimidyl-4-maleimidomethyl cyclohexane-1-carboxylic acid, N-succinimidyl-4-maleimidomethyl benzoic acid, N-succinimidyl-3-maleimidobenzoic acid, N-succinimidyl-4-maleimidophenyl-4-butyric acid, N-succinimidyl-4-maleimidophenyl-4-butyric acid, N,N′-oxydimethylene-dimaleimide, N,N′-o-phenylene-dimaleimide, N,N′-m-phenylene-dimaleimide, N,N′-p-phenylene-dimaleimide, and N,N′-hexamethylene-dimaleimide, N-succinimidyl maleimide carboxylic acid, and the like. As the commercially available product of the maleimide group-containing compound, SANFEL BM-G (manufactured by SANSHIN CHEMICAL INDUSTRY Co., Ltd) can be mentioned. Examples of oxazoline group-containing compounds include oxazoline compounds such as: 2,2′-bis-(2-oxazoline), 2,2′-methylene-bis-(2-oxazoline), 2,2′-ethylene-bis-(2-oxazoline), 2,2′-trimethylene-bis-(2-oxazoline), 2,2′-tetramethylene-bis-(2-oxazoline), 2,2′-hexamethylene-bis-(2-oxazoline), 2,2′-octamethylene-bis-(2-oxazoline), 2,2′-ethylene-bis-(4,4′-dimethyl-2-oxazoline), 2,2′-p-phenylene-bis-(2-oxazoline), 2,2′-m-phenylene-bis-(2-oxazoline), 2,2′-m-phenylene-bis-(4,4′-dimethyl-2-oxazoline), bis-(2-oxazolinylcyclohexane)sulfide, and bis-(2-oxazolinylnorbornane)sulfide. Further, examples of addition polymerizable oxazoline compounds include: 2-vinyl-2-oxazoline, 2-vinyl-4-methyl-2-oxazoline, 2-vinyl-5-methyl-2-oxazoline, 2-isopropenyl-2-oxazoline, 2-isopropenyl-4-methyl-2-oxazoline, 2-isopropenyl-5-ethyl-2-oxazoline, and the like. The compounds obtained by polymerization or copolymerization of one or more of these compounds can also be used. The oxazoline group-containing compounds are commercially available under the names of: Epocros WS-500, Epocros WS-700, Epocros K-1010E, Epocros K-1020E, Epocros K-1030E, Epocros K-2010E, Epocros K-2020E, Epocros K-2030E, Epocros RPS-1005 and Epocros RAS-1005 (all of the above are manufactured by NIPPON SHOKUBAI Co., Ltd.); and NK linker FX (manufactured by SHIN-NAKAMURA CHEMICAL Co., Ltd.). Specific examples of epoxy group-containing compounds include: sorbitol polyglycidyl ether, polyglycerol polyglycidyl ether, diglycerol polyglycidyl ether, glycerol polyglycidyl ether, trimethylolpropane polyglycidyl ether, ethylene glycol diglycidyl ether, polyethylene glycol diglycidyl ether, propylene glycol diglycidyl ether, polypropylene glycol diglycidyl ether, and the like. Two or more kinds of these compounds can be used in combination. Further, the epoxy group-containing compounds are commercially available under the names of: Denacol® EX-611, Denacol® EX-612, Denacol® EX-614, Denacol® EX-614B, Denacol® EX-512, Denacol® EX-521, Denacol® EX-421, Denacol® EX-313, Denacol® EX-314, Denacol® EX-321, Denacol® EX-810, Denacol® EX-811, Denacol® EX-850, Denacol® EX-851, Denacol® EX-821, Denacol® EX-830, Denacol® EX-832, Denacol® EX-841, Denacol® EX-861, Denacol® EX-911, Denacol® EX-941, Denacol® EX-920, Denacol® EX-145 and Denacol® EX-171 (manufactured by Nagase ChemteX Corporation); SR-PG, SR-2EG, SR-8EG, SR-8EGS, SR-GLG, SR-DGE, SR-4GL, SR-4GLS and SR-SEP (all of the above are trade names, manufactured by Sakamoto Yakuhin Kogyo Co., Ltd.); and Epolite 200E, Epolite 400E and Epolite 400P (all of the above are manufactured by KYOEISHA CHEMICAL Co., LTD). The type of the crosslinking agent is not limited to the above mentioned compounds and commercially available products. The crosslinking agent may also be a compound containing at least one functional group selected from aldehyde group, maleimide group, carbodiimide group, oxazoline group and epoxy group. The form of the crosslinking agent is not limited, either. The crosslinking agent may be in the form of monomer, polymer or the like.

### (Electrically Conductive Particles)

It is preferred that the detection layer further include electrically conductive particles. As the electrically conductive particles, particles of a metal such as gold, platinum, silver or palladium; or a higher-order structure made of a carbon material can be used. The higher-order structure can contain, for example, one or more types of fine particles (carbon fine particles) selected from particles of electrically conductive carbon black, Ketjenblack®, carbon nanotube (CNT) and fullerene.

Further, the surface of the detection layer 2 may be covered with an outer-layer film made of cellulose acetate and the like. Examples of raw materials for the outer-layer film, in addition to cellulose acetate, include: polyurethane, polycarbonate, polymethyl methacrylate, butyl methacrylate, polypropylene, polyether ether ketone, and the like.

### (Method for Preparing Enzyme Electrode)

The above mentioned enzyme electrode A is prepared, for example, as follows. Specifically, a metal layer which functions as the electrode 1 is formed on one surface of the insulating base plate 3. For example, a metal layer having a desired thickness (for example, about 30 nm) is formed by depositing a metallic material, by physical vapor deposition (PVD, for example, sputtering) or chemical vapor deposition (CVD), on one surface of the insulating base plate 3 in the form of a film having a predetermined thickness (for example, about 100 μm). It is also possible to form an electrode layer made of a carbon material, instead of the metal layer. Next, the detection layer 2 is formed on the electrode 1. Specifically, a solution (reagent) containing the enzyme 4, electrically conductive polymer 5, the sugar 6 and the crosslinking agent 7 is prepared. The concentration of the sugar in the solution (reagent) is preferably from 0.1 to 2% by weight, and more preferably 0.2 to 2% by weight. The solution (reagent) is dropped on the surface of the electrode 1. Then the solution (reagent) is allowed to dry and solidify on the electrode 1, to obtain the enzyme electrode A in which the detection layer 2 is formed on the electrode 1.

By using the enzyme electrode according to the present invention, the concentration of the substance to be tested contained in a sample can be measured based on the charge transfer limiting current. The substance to be measured is not particularly limited as long as it can be measured by the measuring method using the enzyme electrode of the present invention. However, the substance to be measured is preferably a substance derived from a living body, which can serve as an index of a disease and/or health status, and examples thereof include glucose, cholesterol, and the like. The sample is not particularly limited as long as it contains the substance to be measured. However, a biological sample, such as blood or urine is preferred.

### (Biosensor)

The enzyme electrode according to the present invention can be used as a biosensor, such as a glucose sensor. The biosensor includes, along with the enzyme electrode of the present invention, an electrode which serves as a counter electrode. As the counter electrode, it is possible to use any electrode which can be generally used as the counter electrode in a biosensor. Examples thereof include: a carbon electrode prepared in the form of a film by screen printing; a metal electrode prepared in the form of a film by physical vapor deposition (PVD, for example, sputtering) or chemical vapor deposition (CVD); and a silver/silver chloride electrode prepared in the form of a film by screen printing. It is also possible to employ a 3-electrode system in which the silver/silver chloride electrode or the carbon electrode prepared in the form of a film by screen printing, or the metal electrode prepared in the form of a film by physical vapor deposition (PVD, for example, sputtering) or chemical vapor deposition (CVD), is used as a reference electrode.

### (Apparatus)

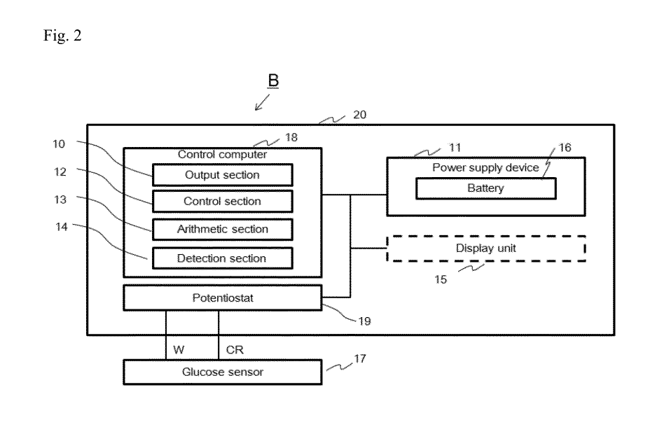

Next, the measuring apparatus according to the present invention will be described with reference to the drawings. Although a glucose measuring apparatus which includes a glucose sensor as the biosensor is illustrated in this embodiment, the measuring apparatus according to the present invention is not limited to the following embodiments. FIG. 2 shows an example of the configuration of main electronic components included in a measuring apparatus B. A control computer 18, a potentiostat 19 and a power supply device 11 are provided on a base plate 20 housed in a housing. The control computer 18 include, as hardware, a processor such as CPU (central processing unit); a recording medium such as a memory (RAM (Random Access Memory) or ROM (Read Only Memory)); and a communication unit. When the processor loads a program stored in the recording medium (for example, the ROM) to the RAM, and executes the program, the control computer 18 functions as an apparatus comprising an output section 10, a control section 12, an arithmetic section 13 and a detection section 14. The control computer 18 may also include an auxiliary memory such as a semiconductor memory (EEPROM or flash memory) or a hard disk.

The control section 12 controls the timing for applying the voltage and the value of the voltage to be applied. The power supply device 11 includes a battery 16, and supplies electricity to the control computer 18 and the potentiostat 19 for operation. It is also possible to dispose the power supply device 11 outside the housing. The potentiostat 19 is a device which maintains the potential of the working electrode constant with respect to the potential of the reference electrode. The potentiostat 19, which is controlled by the control section 12, applies a predetermined amount of voltage between the counter electrode and the working electrode of the glucose sensor 17 using terminals CR and W; measures the response current of the working electrode which can be obtained at the terminal W; and send the result of the measurement to the detection section 14.

The arithmetic section 13 calculates the concentration of the substance to be measured based on the value of the detected current, and stores the calculated result. The output section 10 carries out data communication between the output section 10 and the display section unit 15, and sends the calculated result of the concentration of the substance to be measured provided by the arithmetic section 13 to the display section unit 15. The display section unit 15 is capable of displaying, for example, the calculated result of the glucose concentration which is received from the measuring apparatus B, on a display screen in a predetermined format.

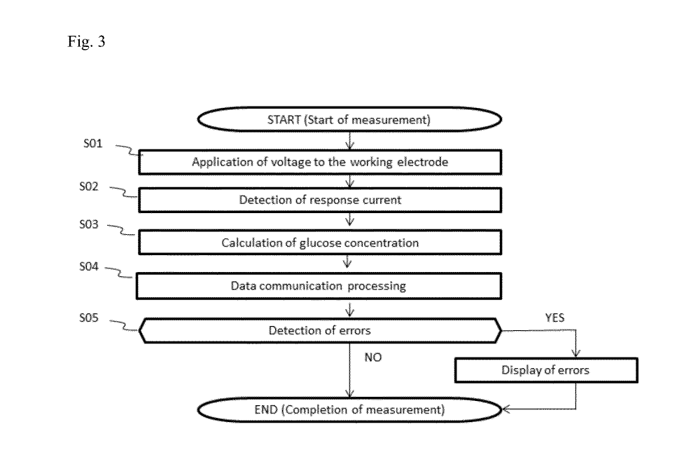

FIG. 3 is a flow chart showing an example of the processing sequence of the glucose concentration measurement carried out by the control computer 18. When the CPU (control section 12) of the control computer 18 receives an instruction to start the measurement of the glucose concentration, the control section 12 controls the potentiostat 19 to apply a predetermined amount of voltage to the working electrode, and starts measuring the response current from the working electrode (Step S01). Further, the detection of the installation of a sensor to the measuring apparatus may be used as the instruction to start the measurement of the concentration.

Next, the potentiostat 19 measures the response current generated by the application of voltage, specifically, the charge transfer limiting current based on the transfer of electrons derived from the substance to be measured (glucose, in this embodiment) in the sample to the electrode, preferably, the steady-state current observed after the occurrence of the transient current due to the charging of an electric double layer, for example, the steady-state current observed 1 to 20 seconds after the application of voltage. Then the potentiostat 19 sends the measured current to the detection section 14 (Step S02).

The arithmetic section 13 carries out arithmetic processing based on the measured current value, and calculates the glucose concentration (Step S03). For example, the formulae for calculating the glucose concentration or the data of the calibration curve of the glucose concentration, which correspond to the glucose dehydrogenase disposed on the electrode, are preinstalled to the arithmetic section 13 in the control computer 3, and the arithmetic section 13 calculates the glucose concentration utilizing these calculation formulae or the calibration curve.

The output section 10 sends the calculated result of the glucose concentration to the display section unit 15, through a communication link provided between the output section 10 and the display section unit 15 (Step S04). Thereafter, the control section 12 determines if there are any measurement errors detected (Step S05); completes the measurement if there is no error; and displays the glucose concentration on the display section. If there are any errors, a notification of error is displayed, and then the flow sequence shown in FIG. 3 is completed. Further, the calculated result can be stored in the arithmetic section 13, so that the stored result can be reloaded afterwards to be displayed on the display section for confirmation. Although the detection of measurement errors by the control section 12 (Step S05) is carried out after the calculated result is sent to the display section unit 15 (Step S04) in this embodiment, it is also possible to carry out these steps in different orders.

### EXAMPLES

Examples of the enzyme electrode will now be described.

### (Test 1)

### (Preparation of Reagent Solution)

Two types of reagent solutions according to Example 1 and Comparative Example 1 as described below were prepared.

### Example 1

Ketjenblack® (Mitsubishi Carbon Black): 1.20%

Electrically conductive polymer: sulfonated polyaniline aqueous solution (trade name: aquaPASS-01x; manufactured by Mitsubishi Rayon Co., Ltd.): 0.40% Oxazoline group-containing polymer, Epocros WS-700 (manufactured by NIPPON SHOKUBAI Co., Ltd.): 6.0%

Enzyme (Cy-GDH): 4.5 mg/mL

Sugar (sucrose): 0.50%

Phosphate buffer solution (pH 5.8): 10 mM

Note that, “%” represents the percent by weight concentration of the reagent contained in the reagent solution.

### Comparative Example 1

Ketjenblack® (Mitsubishi Carbon Black): 1.20%

Electrically conductive polymer: sulfonated polyaniline aqueous solution (trade name: aquaPASS-01x; manufactured by Mitsubishi Rayon Co., Ltd.): 0.40% Oxazoline group-containing polymer, Epocros WS-700 (manufactured by NIPPON SHOKUBAI Co., Ltd.): 6.0%

Enzyme (Cy-GDH): 4.5 mg/mL

Phosphate buffer solution (pH 5.8): 10 mM

where “%” represents the percent by weight concentration of the reagent contained in the reagent solution. As can be seen from the above, the reagent solution according to Comparative Example 1 has the same composition as the reagent solution according to the Example 1, except that it contains no sugar.

### (Preparation of Enzyme Electrode (Sample)

Next, a plurality of insulating base plates each having on one surface thereof an electrode (electrode layer) formed by gold vapor deposition (base materials) were prepared. The reagent solution according to Example 1 or Comparative Example 1 was dispensed on each of the insulating base plates, and the resulting base materials were dried by allowing them to stand for 30 minutes in a low humidity drying furnace. The reagents were thus allowed to solidify on respective electrodes to form detection layers, to obtain enzyme electrodes (samples) according to Example 1 and enzyme electrodes (samples) according to Comparative Example 1, each having a detection layer formed on one surface thereof.

### (Measurement of Glucose Concentration)

Next, the response current value was measured for a human whole blood sample in which the concentration of glucose was adjusted to 343 mg/dL (sample: S); or a human whole blood sample in which the concentration of glucose was adjusted to 0 mg/dL (blank: B), using each of the enzyme electrodes (working electrodes). The glucose measurement was carried out using a reference electrode/counter electrode (both made of carbon) and a reference electrode (silver/silver chloride electrode) formed on the electrode base plate, with a voltage of +0.2 V applied to the working electrode (vs. Ag/AgCl).

### (Evaluation of Measurement Results)

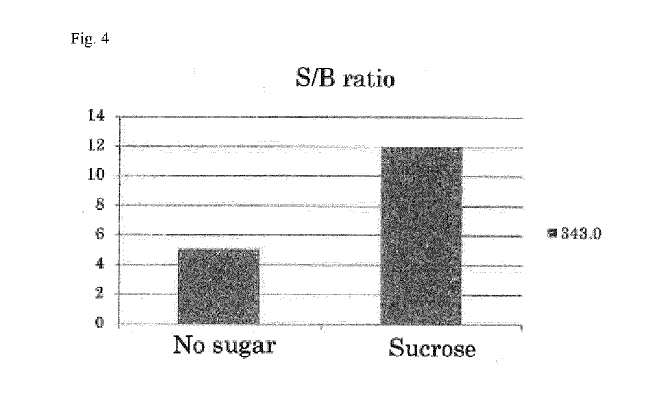

FIG. 4 is a graph showing the S/B ratio of the current values obtained by using the enzyme electrodes according to Comparative Example 1 and Example 1. Based on the test results shown in FIG. 4, it can be seen that the detection sensitivity is improved when the enzyme electrodes according to Example 1 were used, in which each of the detection layers contained a sugar, compared to the cases where the enzyme electrodes according to Comparative Example 1 were used, in which each of the detection layers contained no sugar.

### Comparative Example 2

Enzyme electrodes each comprising on one surface thereof a detection layer formed using a reagent solution having the following composition were prepared, in the same manner as described above.

Ketjenblack® (Mitsubishi Carbon Black): 1.20%

Electrically conductive polymer: sulfonated polyaniline aqueous solution (trade name: aquaPASS-01x; manufactured by Mitsubishi Rayon Co., Ltd.): 0.40%

Enzyme (Cy-GDH): 4.5 mg/mL

Sugar (sucrose): 0.50%

Phosphate buffer solution (pH 5.8): 10 mM

Note that, “%” represents the percent by weight concentration of the reagent contained in the reagent solution. As can be seen from the above, the reagent solution according to Comparative Example 2 has the same composition as the reagent solution according to Example 1, except that it contains no crosslinking agent.

The response current value was measured for an aqueous solution containing 343 mg/dl of glucose (sample: S) or for water (blank: B), using the enzyme electrodes according to Comparative Example 2. As a result, even in the case where the aqueous solution of glucose was used, the current was barely detectable, and it was impossible to measure the glucose concentration.

### (Test 2)

Enzyme electrodes each comprising a detection layer having the same composition as those prepared in Example 1 except for containing one of various types of sugar instead of sucrose were prepared. Then, using the thus prepared enzyme electrodes, the response current value was measured for a human whole blood sample whose glucose concentration was adjusted to 256 mg/dl or to 512 mg/dl (sample: S); or a human whole blood sample whose glucose concentration was adjusted to 0 mg/dL (blank: B), and the S/B ratio was obtained for each of the samples, in the same manner as in Example 1.

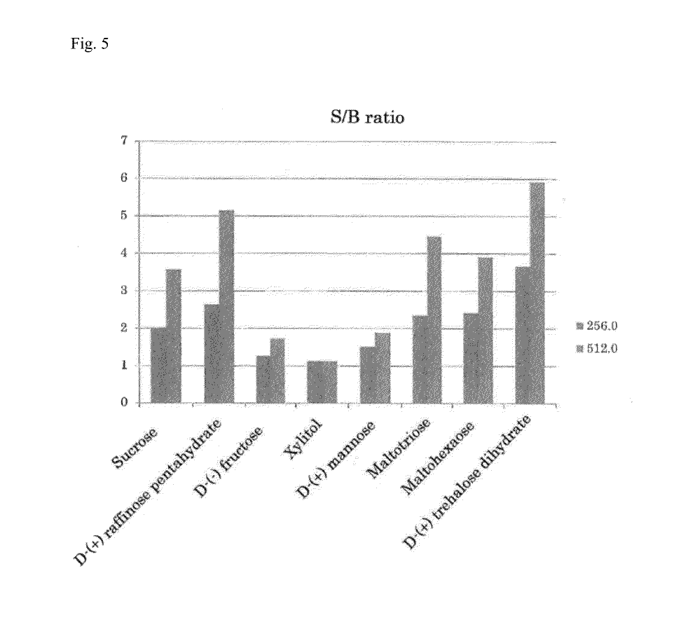

FIG. 5 is a graph showing the results of the test 2, specifically, a graph of the S/B ratio of the current obtained by using the enzyme electrodes each comprising a detection layer containing one of various types of sugar. According to the test results shown in FIG. 5, the S/B ratio was low when the enzyme electrodes each comprising a detection layer containing xylitol, mannose or fructose were used, and the concentration dependence was barely observed. However, when the enzyme electrodes each comprising a detection layer containing trehalose, sucrose, maltotriose, raffinose or maltohexaose were used, the S/B ratio was improved, and the concentration dependence for glucose was observed. These results have revealed that the incorporation of a sugar in the detection layer of the enzyme electrode allows for an improvement in the detection sensitivity of the enzyme electrode, and for a quantitative measurement of the substance to be measured.

### DESCRIPTION OF SYMBOLS

A enzyme electrode
1 electrode
2 detection layer
3 insulating base plate
4 enzyme
5 electrically conductive polymer
6 sugar
7 crosslinking agent (main chain)
7′ crosslinking agent (side chain)
B measuring apparatus
10 output section
11 power supply device
12 control section
13 arithmetic section
14 detection section
15 display section unit
16 battery
17 glucose sensor
18 control computer
19 potentiostat
20 base plate
CR, W terminals

While the invention has been described in detail with reference to preferred embodiments thereof, it will be apparent to one skilled in the art that various changes can be made, and equivalents employed, without departing from the scope of the invention. Each of the aforementioned documents as well as JP2014-261203 is incorporated by reference herein in its entirety.

What is claimed is:

1. An enzyme electrode, comprising:
an electrode; and
a detection layer in contact with the electrode, and comprising an enzyme, a crosslinking agent, an electrically conductive polymer and a sugar;
wherein electrons are transferred between the enzyme in the detection layer and the electrode.

2. The enzyme electrode according to claim 1, wherein the sugar is at least one sugar containing 2 or more monosaccharide units.

3. The enzyme electrode according to claim 1, wherein the sugar is a disaccharide, a trisaccharide, a tetrasaccharide, a pentasaccharide or a hexasaccharide.

4. The enzyme electrode according to claim 1, wherein the sugar is trehalose, sucrose, maltotriose, raffinose or maltohexaose.

5. The enzyme electrode according to claim 1, wherein the sugar is a sugar which does not serve as a substrate for the enzyme.

6. The enzyme electrode according to claim 1, wherein the detection layer does not contain an electron mediator.

7. The enzyme electrode according to claim 1, wherein the enzyme comprises molecules that are crosslinked by the crosslinking agent.

8. The enzyme electrode according to claim 1, wherein the detection layer further comprises electrically conductive particles.

9. The enzyme electrode according to claim 1, wherein the enzyme is an oxidoreductase.

10. The enzyme electrode according to claim 9, wherein the oxidoreductase is a saccharide oxidoreductase selected from glucose oxidase, galactose oxidase, glucose dehydrogenase, fructose dehydrogenase and sorbitol dehydrogenase.

11. The enzyme electrode according to claim 1, wherein the crosslinking agent is a compound containing at least one functional group selected from an aldehyde group, a maleimide group, a carbodiimide group, an oxazoline group and an epoxy group.

12. The enzyme electrode according to claim 1, wherein the electrically conductive polymer is selected from the group consisting of polypyrrole, polyaniline, polystyrene sulfonate, polythiophene, polyisothianaphthene, polyethylenedioxythiophene(poly(3,4-ethylenedioxythiophene)poly(styrene sulfonate)), and combinations thereof.

13. A biosensor comprising the enzyme electrode according to claim 1.

14. A biosensor according to claim 13 which further comprises a counter electrode.

15. A measuring apparatus for measuring the concentration of a substance in a sample comprising:
the biosensor according to claim 13;
a control section configured to control application of voltage to the biosensor;
a detection section configured to detect a charge transfer limiting current based on transfer of electrons from said substance to the electrode, wherein the charge transfer limiting current is generated by application of voltage to the biosensor;
an arithmetic section configured to calculate concentration of the substance based on the value of the charge transfer limiting current; and
an output section configured to output the calculated concentration of the substance.

16. A method for producing an enzyme electrode according to claim 1 comprising forming a detection layer on an electrode by applying a mixed solution on the electrode, wherein the mixed solution comprises an enzyme, a crosslinking agent, an electrically conductive polymer and a sugar; and wherein electrons are transferred between the enzyme and the electrode in the enzyme electrode.
